(19) United States
(12) Patent Application Publication
(10) Pub. No.: US 2008/0172253 A1
(43) Pub. Date: Jul. 17, 2008

(54) SYSTEM AND METHOD FOR ADMINISTERING MEDICATION

(75) Inventors: Wai Yee Joanne Chung, Hung Hom (HK); Kwok Shing Thomas Wong, Hung Hom (HK)

Correspondence Address: BLAKELY SOKOLOFF TAYLOR & ZAFMAN, 1279 OAKMEAD PARKWAY, SUNNYVALE, CA, 94085-4040, US

(73) Assignee: The Hong Kong Polytechnic University, Hung Hom (HK)

(21) Appl. No.: 11/622,311

(22) Filed: Jan. 11, 2007

(51) Int. Cl. A61B 5/02

(52) U.S. Cl. 705/3; 700/15; 700/244; 705/23; 705/29

(57) ABSTRACT

An administration system (10) for administering medication (25) to peoples the system comprising: a medicine cabinet (20) to contain a plurality of medication (25), each medication (25) identified by at least one radio frequency identification (RFID) tag (26); at Feast one RFID reader (40) to monitor the storage and removal of medication (25) from the medicine cabinet (20); a database (70) to store information related to the medication (25); an alert module (35) to provide an alert to the person if incorrect medication (25) have been removed from the medicine cabinet (20) or if correct medication (25) have not been removed from the medicine cabinet (20) at the correct time, wherein the information stored by the database (70) includes any one from the group consisting of: contraindications combination of medication (25) which may be safely taken, allergic reactions of medication (25), the recommended dosage for each medication (25), the administration schedule for each medication (25), and expiry date of each medication (25).

### TECHNICAL FIELD

The invention concerns an administration system for administering medication to patients.

### BACKGROUND OF THE INVENTION

In an ageing population, there is a high demand for doctors and healthcare professionals to administer high quality levels of healthcare services to the general public.

However, health care facilities and manpower are limited and are unable to satisfy the increasing demand. Introducing information technology to healthcare services may improve the productivity of health care professionals and allow information to be efficiently shared. Medical records are valuable to both healthcare professionals and patients because the medical records reveal history and clues on the progress of the health of patient's. A drug taking log forms part of these medical records and should ideally be well organized to document the patients' medicine taking details.

Problems associated with drug taking may be incorrect medicine taking or improper dosage. This problem is especially likely for older patients or someone who is illiterate. They may forget the drug taking instructions or take another patient's drug mistakenly. Also, regular health assessments help patients to take drugs with close monitoring on their vital signs.

Accordingly, there is a desire for an administration system that alleviates at least some of the abovementioned areas of concern.

### SUMMARY OF THE INVENTION

In a first preferred aspect, there is provided an administration system for administering medication to people, the system comprising:

a medicine cabinet to contain a plurality of medication, each medication identified by at least one radio frequency identification (RFID) tag;

at least one RFID reader to monitor the storage and removal of medication from the medicine cabinet;

a database to store information related to the medication;

an alert module to provide an alert to the person if incorrect medication have been removed from the medicine cabinet or if correct medication have not been removed from the medicine cabinet at the correct time;

wherein the information stored by the database includes any one from the group consisting of contraindications, combination of medication which may be safely taken, allergic reactions of medication, the recommended dosage for each medication, the administration schedule for each medication, and expiry date of each medication

The system may further comprise at least one measurement device to measure at least one characteristic of the person, the measured characteristic is considered by the alert module to determine whether a medication should be taken by the person. The at least one characteristic of the person may be any one from the group consisting of: blood pressure, body temperature. ECG and blood glucose level.

The alert module may comprise a communications module to transmit a message to at least one predetermined mobile communications device, the message informing the message recipient that the person has removed incorrect medication from the medicine cabinet or correct medication have not been removed from the medicine cabinet at the correct time.

The database may further store person related information, RFID tag identifiers and data collected from the at least one RFID reader.

The system may further comprise a display device operatively connected to the medicine cabinet to display images of the medication to be taken by the person and the prescribed dosage for the person.

The system may further comprise a speaker operatively connected to the medicine cabinet to playback audio recordings of descriptions of medication to be taken by the person and the prescribed dosage for the person.

The medication may include both Western pharmaceuticals and Chinese herbs.

In a second aspect, there is provided a computer-implemented method for administering medication to people, the method comprising:

monitoring storage and removal of medication from a medicine cabinet, each medication identified by at least one radio frequency identification (RFID) tag;

comparing information retrieved from the RFID tags against information related to the medication stored in a database;

alerting the person if incorrect medication have been removed from the medicine cabinet or if correct medication have not been removed from the medicine cabinet at the correct time;

wherein the information stored by the database includes any one from the group consisting of contraindications, combination of medication which may be safely taken, allergic reactions of medication, the recommended dosage for each medication, the administration schedule for each medication, and expiry date of each medication.

In a third aspect, there is provided a medicine cabinet for administering medication to people, the cabinet comprising:

a storage area to store medication, each medication identified by at least one radio frequency identification (RFID) tag;

at least one RFID reader to monitor the storage and removal of medication from the medicine cabinet;

wherein the RFID reader reads the RFID tags of the medication and transmits the information to an alert module to provide an alert to the person if incorrect medication have been removed from the medicine cabinet or if correct medication have not been removed from the medicine cabinet at the correct time by comparing RFID tags read by the RFID reader against information stored in a database, the information stored in the database including any one from the group consisting of contraindications, combination of medication which may be safely taken, allergic reactions of medication, the recommended dosage for each medication, the administration schedule for each medication, and expiry date of each medication.

In a fourth aspect, there is provided a medication comprising:

at least one radio frequency identification (RFID) tag;

wherein the RFID tag is read by an RFID reader to obtain identification information related to the medication and transmit the identification information to an alert module to provide an alert to the person if incorrect medication have been removed from the medicine cabinet or if correct medication have not been removed from the medicine cabinet at the correct time by comparing the RFID tag read by the RFID reader against information stored in a database, the information stored in the database including any one from the group consisting of: contraindications, combination of medication which may be safely taken, allergic reactions of medication, the recommended dosage for each medication the administration schedule for each medication, and expiry date of each medication.

### BRIEF DESCRIPTION OF THE DRAWINGS

An example of the invention will now be described with reference to the accompanying drawings, in which:

FIG. 1 is a block diagram of an administration system for administering medication to patients according to a preferred embodiment of the present invention; and

FIG. 2 is a process flow diagram showing a checking algorithm of a medication administration method according to an embodiment of the present invention.

### DETAILED DESCRIPTION OF THE DRAWINGS

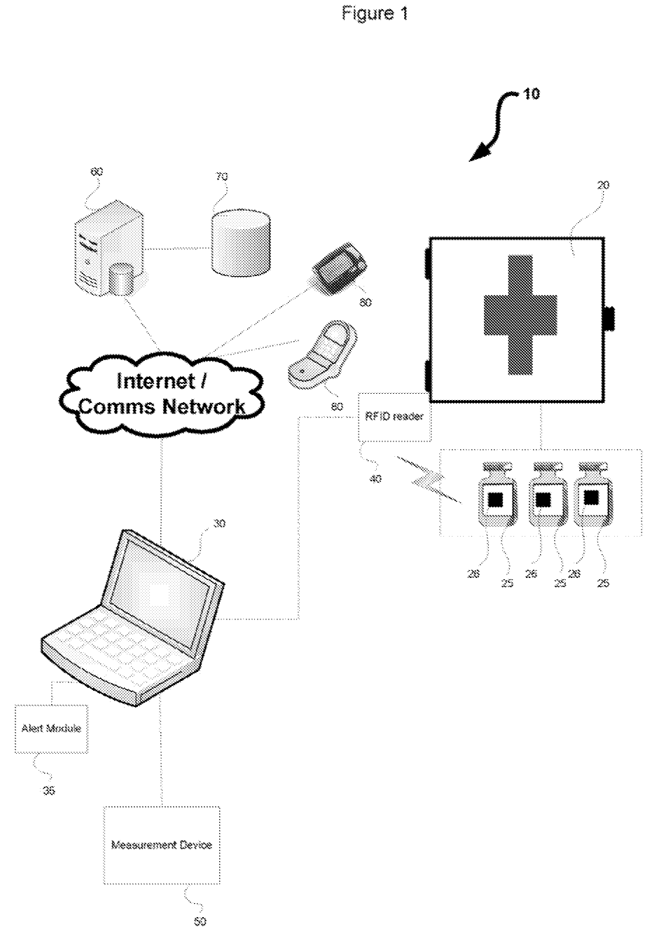

Referring to FIG. 1, there is provided an administration system 10 for administering medication 25 to patients. The system 10 generally comprises: a medicine cabinet 20, at least one RFID reader 40, a database 70, and an alert module 35. The medicine cabinet 20 contains a plurality of medication 25. Each medication 25 is identified by at least one radio frequency identification (RFID) tag 26. The RFID reader 40 monitors the storage and removal of medication 25 from the medicine cabinet 20 through detection of the RFID tags 26. The database 70 stores information related to the medication 25. The alert module 35 provides an alert to the patient if incorrect medication 25 has been removed from the medicine cabinet 20 or if correct medication 25 has not been removed from the medicine cabinet 20 at the correct time. The information stored by the database 70 includes any one from the group consisting of: contraindications, combination of medication 25 which may be safely taken, allergic reactions of medication 25, the recommended dosage for each medication 25, the administration schedule for each medication 25, and expiry date of each medication 25.

Data regarding the use of the medication 25 are input into the database 70 by a health care professional. The database 70 may reside on either the server side or client side. If there is a local database 70 on the client side, there may be periodic synchronization with a master database located on the server side. A centralized database on the server side is accessible by healthcare professionals remotely via the Internet. Use of the REID tag 26 enables the medication 25 to be tracked whenever they are taken out or placed back into the medicine cabinet 20. The tracking and tracing data of the medication 25 is also stored in the database 70.

The system 10 also includes a user-friendly screen interface 30 for the patient featuring a visual display and a computer-synthesized voice option. The screen interface 30 may also be a touch screen panel. Reminders can be set to alert and assist the patients to take the right medicine 25 at the right time. The visual display and speaker for the voice option may be provided on a computer 30 operatively attached to the medicine cabinet 20, or may be integrated into the structural body of the medicine cabinet 20. The computer 30 may be a notebook computer or a handheld computer. If integrated into the medicine cabinet 20, the visual display is situated on the front panel for the patient to see.

The RFID reader 40 is connected to the computer 30 wirelessly or via a cable. The RFID reader 40 is physically mounted or attached to the medicine cabinet 20. The RFID reader 40 reads and collects data from the REID tags 26 regarding the administration of the medication stored in the medicine cabinet 20. Data collected by the RFID reader 40 is sent to the computer 30. The computer 30 then transmits the data to a database 70 through an off site server 60 via the Internet. All the warning generation, reminder checking and processing are conducted by the alert module 35 running on the computer 30. The alert module 35 communicates with the server 60 which interrogates the database 70 for data relating to the patient and the patient's medication 25. Once data is received from the server 60, the alert module 35 processes the data and considers other factors such as the current date and time and any measurements taken by measurement device, to decide whether an alert or reminder needs to be generated for the patient.

As part of a holistic system, a physical measurement device 50 is provided to measure the physical condition of patients, such as blood pressure, body temperature, ECG or and blood glucose level, if required, before taking any medication. For certain illnesses, medication may not be required if the patient's blood pressure is within a prescribed safe range. The measurement device 50 is connected to the medicine cabinet 20 or the computer 30 via connectors. Several types of connectors are provided to enable various kinds of measurement devices 50 to be used.

The system 10 is installed in the patient's home or office and is connected to the Internet to enable healthcare professionals to identify usage of the medication 25 stored within the medicine cabinet 20. Also, as there is a detailed log of medication 25 taken by the patient, healthcare professionals are able to gain a higher degree of certainty on what has been taken by the patient over a long period of timer the administration regime used, and also whether the treatment programme has been effective in treating the patient's medical problems.

The system 10 also enables transmission of alerts to mobile communication devices such as pagers, mobile phones and handheld computers. The alert module 35 is configured to store pager numbers, mobile phone numbers, e-mail addresses of people to send an alert to if the patient does not take a medication 25 they are supposed to at a prescribed time, or takes a medication 25 they are not supposed to. The people that the alert is typically transmitted to are: the patient's doctor, other healthcare professionals and family members. Once they receive an alert, they may contact the patient via telephone, or visit the patient in person, if necessary.

The system 10 facilitates remote monitoring of patient's consumption of medicine 25 by having the medicine cabinet 20 placed in the patient's home or workplace. This allows healthcare professionals to analyze the medicine-taking pattern and detect any wrong taking of medicine 25 at an early point in time. The system 10 provides a means for enabling healthcare professionals to monitor and assist their remotely located patients to administer the correct and suitable medication 25 by utilizing the latest telecommunication technology. The system 10 provides a user-friendly interface with a visual display of reminders and computer-synthesized voice or alarm to help patients self-administer their medications 25.

The system 10 is especially helpful for the elderly people who live in remote locations or for visually impaired patients who have difficulty following written instructions when administering medicine 25.

The alert module 35 complies with the “3 Checks 5 Rights” principle. The three checks related to before administration of the medication and are completed when retrieving medication, after preparing medication and prior to medication administration. The five rights are: right patient/client, right drug, right route, right dose, and right time. Right patient means identifying the correct patient, Right drug means checking record for name of drug and compare with drug on hand. Right route means checking medication record for how to administer the drug and check labeling of drug to ensure it matches prescribed route. Right dose means comparing ordered dose to dose on hand. At times, calculations may need to be performed to ascertain the correct dose. For example, a scored tablet, or one that is designed and intended for dividing, may need to be halved or quartered in order to administer the correct oral dose. Right time means verifying that frequency or time ordered matches current time. This is a principle of medicine dispensing in the healthcare discipline. Whenever the system 10 detects that a patient is taking a medication 25 from the cabinet 20, the following rules are checked:
1) If taking a medicine 25 would be harmful for a patient who is not in a good physical condition, such as relatively low blood pressure, or relatively slow heart rate, the system 10 reminds the patient to take a measurement with an attached self-operated measuring device 50, for example to measure their blood pressure.
2) If a patient picks up a medicine 25 which does not belong to him or her, the system 10 warns the patient.
3) If a patient picks up a medicine 25 at the wrong time, the system 10 warns the patient.
4) If a patient is known to have an allergy to a medicine 25 that he or she picks up from the cabinet 20, the system 10 warns the patient.
5) If the medicine 25 picked up by the patient has contraindication to another medicine they are taking, the system 10 warns the patient.
6) An instruction reminder is sent to the patient on the dosage and administration route of the medicine to be taken.

Whenever there is a violation of any of these rules, the system 10 prompts the patient with an audible alarm and/or a visual message with the relevant indication of the problem on a screen on the cabinet 20 or computer 30. After the warning or reminder, the patient is expected to stop taking the wrong medicine and seek advice from a healthcare professional as soon as possible.

The system 10 provides an efficient and remote way for dispensing medicine 25 at the patient's own home or workplace and also detects any misuse of medication consumption. For the patient, the system 10 provides user-friendly visual and audible features for self-administration of medicine, especially elderly or visually impaired patients. Real time warnings and reminders on the use of medicine 25 are sent to patients to avoid incorrect medicine taking. For healthcare professionals, with regard to the concept of telemedication management, the tracking is stored in the database 70, allowing the data to be retrieved remotely in real time by a healthcare professional for monitoring or analyzing. The tracking data is used by healthcare professionals to investigate or improve the patient's medicine taking. The system 10 interfaces with the telemedication management system to establish a holistic tele-based medication management system for physical checking and administration of medicine over the Internet.

The system 10 makes use of the real-time medication data captured from the RFID technology-based tag 26 to provide assistance of medication for the remote patients by means of real-time warnings and reminders.

The design of the database 70 stores information about Western medicine and Chinese herbs Checking for contraindication covering both categories of medicine/herbs is also performed by the alert module 35.

The system 10 also connects to a telehealth system through a standard interface on the software platform. It allows direct access to any real-time and historical healthcare-related data of the patients using the system 10.

The system 10 reduces the risk of human error occurring during the medication of patients, especially for patients with difficulties in following the healthcare professionals' instructions, such as the elderly and visually impaired patients.

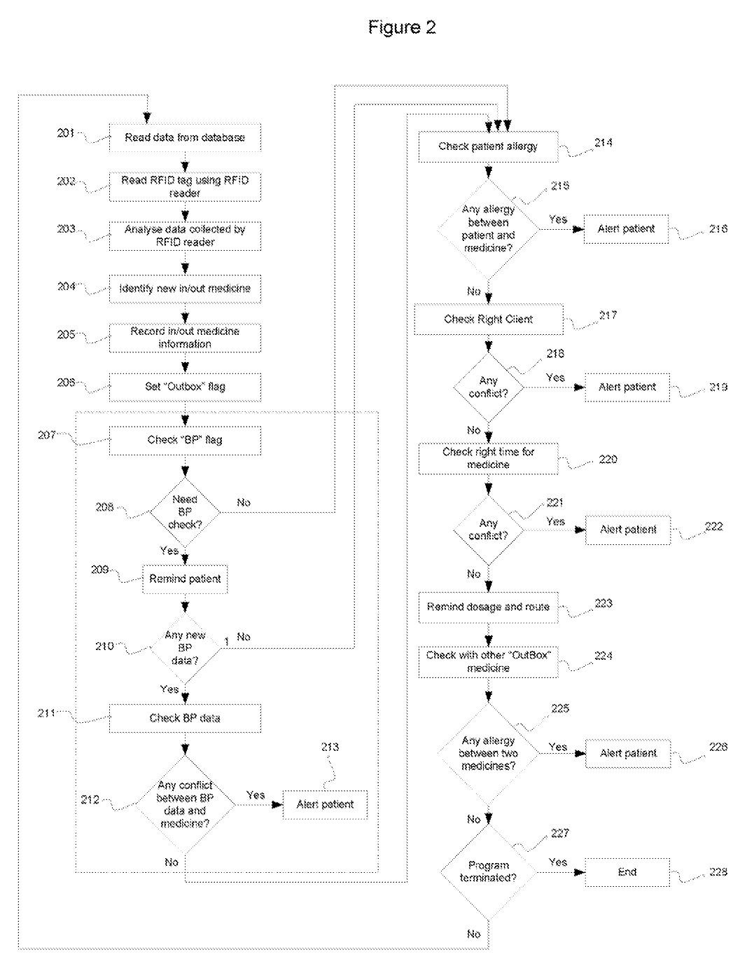

Referring to FIG. 2, a computer-implemented method for administering medication to patients is provided. Typically, the alert module 35 performs a read 201 of the database 70 by initiating a connection over the Internet via the server 60. When medication 225 are removed or placed into the medicine cabinet 20, the RFID tag 26 is read 202 by the RFID reader 40. The alert module 35 analyses 203 the RFID data collected by the RFID reader 40. Any movement of medication 25 to and from the medicine cabinet 20 is identified 204 and recorded 205. An “Outbox” flag is set 206. The next series of steps is referred to as the physical measurement phase. In the physical measurement phase, the “BP” flag is checked 207 to determine whether a measurement of the blood pressure of a patient needs to conducted due to taking of a specific medication 25 or due to the patient's circumstances. If the patient's blood pressure requires a measurement 208, the patient is reminded 209 to take this measurement. If there is new blood pressure data 210, the new blood pressure data is checked 211. The alert module 35 then determines 212 whether any conflict exists between the new blood pressure data and the medication 25. If there is, the patient is alerted 213. Next, the allergies of the patient are checked 214. If there is an allergy between the patient and a medication 25, the patient is alerted 216. Next, the right client is checked to determine the patient's identity in cases where the medicine cabinet 20 is used by more than one patient. This is to ensure that only correct medication 25 is consumed by the correct patient. Checking the identity may be performed by thumbprint scan, retina scan, voice analysis, or simply touching the screen to indicate to the system 10 who the patient is. If the patient attempts to take a medication 25 that does not belong to him or her, an alert is generated 219. Next, the right time for taking the medicine 25 is checked 220. The alert module 35 compares the current date and time against the database 70 to determine whether it is now the correct time to take the medicine 25 by the patient. If it is not the correct time, an alert is generated 222. The alert module 35 also reminds 223 the patient of the dosage and administration route. This may be visual images and text on the display of the medicine cabinet 20 and/or also audio instructions. Other “OutBox” medicine is also checked 224. The alert module 35 also checks 225 if there is any allergies between two or more medication 25. If there is, an alert is generated 226. The alert module 35 checks whether the process is to be terminated. Conditions which determine whether the process is terminated may be if the patient closes the medicine cabinet 20, or touches the display to indicate the process has ended 228. If the process is to be continued because more medication 25 are removed or placed into the medicine cabinet 20 the process begins again.

It will be appreciated by persons skilled in the art that numerous variations and/or modifications may be made to the invention as shown in the specific embodiments without departing from the scope or spirit of the invention as broadly described. The present embodiments are, therefore, to be considered in all respects illustrative and not restrictive.

We claim:

1. An administration system for administering medication to people, the system comprising:
a medicine cabinet to contain a plurality of medication, each medication identified by at least one radio frequency identification (RFID) tag;
at least one RFID reader to monitor the storage and removal of medication from the medicine cabinet;
a database to store information related to the medication;
an alert module to provide an alert to the person if incorrect medication have been removed from the medicine cabinet or if correct medication have not been removed from the medicine cabinet at the correct time;
wherein the information stored by the database includes any one from the group consisting of contraindications, combination of medication which may be safely taken, allergic reactions of medication, the recommended dosage for each medication, the administration schedule for each medication, and expiry date of each medication

2. The system according to claim 1, further comprising at least one measurement device to measure at least one characteristic of the person, the measured characteristic is considered by the alert module to determine whether a medication should be taken by the person.

3. The system according to claim 1, wherein the alert module comprises a communications module to transmit a message to at least one predetermined mobile communications device, the message informing the message recipient that the person has removed incorrect medication from the medicine cabinet or correct medication have not been removed from the medicine cabinet at the correct time.

4. The system according to claim 1, wherein the database further stores person related information, RFID tag identifiers and data collected from the at least one RFID reader.

5. The system according to claim 2, wherein the at least one characteristic of the person is any one from the group consisting of: blood pressure, body temperature, ECG and blood glucose level.

6. The system according to claim 1, further comprising a display device operatively connected to the medicine cabinet to display images of the medication to be taken by the person and the prescribed dosage for the person.

7. The system according to claim 1, further comprising a speaker operatively connected to the medicine cabinet to playback audio recordings of descriptions of medication to be taken by the person and the prescribed dosage for the person.

8. The system according to claim 1, wherein the medication include both Western pharmaceuticals and Chinese herbs.

9. A computer-implemented method for administering medication to people, the method comprising:
monitoring storage and removal of medication from a medicine cabinet, each medication identified by at least one radio frequency identification (RFID) tag;
comparing information retrieved from the RFID tags against information related to the medication stored in a database;
alerting the person if incorrect medication have been removed from the medicine cabinet or if correct medication have not been removed from the medicine cabinet at the correct time;
wherein the information stored by the database includes any one from the group consisting of: contraindications, combination of medication which may be safely taken, allergic reactions of medication, the recommended dosage for each medication, the administration schedule for each medication, and expiry date of each medication.

10. A medicine cabinet for administering medication to people, the cabinet comprising:
a storage area to store medication, each medication identified by at least one radio frequency identification (RFID) tag,
at least one RFID reader to monitor the storage and removal of medication from the medicine cabinet;
wherein the RFID reader reads the RFID tags of the medication and transmits the information to an alert module to provide an alert to the person if incorrect medication have been removed from the medicine cabinet or if correct medication have not been removed from the medicine cabinet at the correct time by comparing RFID tags read by the RFID reader against information stored in a database, the information stored in the database including any one from the group consisting of: contraindications, combination of medication which may be safely taken, allergic reactions of medication, the recommended dosage for each medication, the administration schedule for each medication, and expiry date of each medication.

11. A medication comprising:
at least one radio frequency identification (RFID) tag;
wherein the RFID tag is read by an RFID reader to obtain identification information related to the medication and transmit the identification information to an alert module to provide an alert to the person it incorrect medication have been removed from the medicine cabinet or if correct medication have not been removed from the medicine cabinet at the correct time by comparing the RFID tag read by the RFID reader against information stored in a database, the information stored in the database including any one from the group consisting of: contraindications, combination of medication which may be safely taken, allergic reactions of medication, the recommended dosage for each medication, the administration schedule for each medication, and expiry date of each medication.
